(19) United States
(12) Patent Application Publication
(10) Pub. No.: US 2011/0262969 A1
(43) Pub. Date: Oct. 27, 2011

(54) METHOD FOR PRODUCING OPTICALLY ACTIVE, CYCLIC DEPSIPEPTIDES COMPRISING LACTIC ACID AND PHENYL LACTIC AND HAVING 24 RING ATOMS, USING FUNGUS STRAINS OF ROSELLINIA TYPE, AND FURTHER SPECIES OF XYLARIACEAE

(76) Inventors: Achim Harder, Koln (DE); Thi Lam Huong Pham, Berlin (DE); Rene Jarling, Berlin (DE); Irmtraut Zaspel, Muncheberg (DE); Dietrich Ewald, Golsdorf (DE)

(21) Appl. No.: 13/133,611

(22) PCT Filed: Dec. 8, 2009
(86) PCT No.: PCT/EP09/08740

§ 371 Date: Jul. 18, 2011

(30) Foreign Application Priority Data
Dec. 16, 2008 (EP) 08021792.0

(51) Int. Cl. C12P 21/04; C12P 13/04

(52) U.S. Cl. 435/71.1; 435/106

(57) ABSTRACT

The present invention relates to a process for the preparation of lactic- and phenyllactic-acid-containing, optically active, cyclic depsipeptides with 24 ring atoms by means of fungal strains of the genus Rosellinia and further Xylariaceae isolated not only from fruiting bodies of Rosellinia spp. and Xylariaceae growing on dead timber and live timber of deciduous and coniferous trees, but also directly from timber and roots of deciduous and coniferous trees, or by means of enzymatic preparations isolated from these fungal strains. PF1022A, of the general formula (I), is outstandingly suitable for controlling endoparasites, in particular in the field of medicine and veterinary medicine.

Process for the preparation of lactic- and phenyllactic-acid-containing, optically active, cyclic depsipeptides with 24 ring atoms with the aid of fungal strains of Rosellinia species and further Xylariaceae.

The present invention relates to a process for the preparation of lactic- and phenyllactic-acid-containing, optically active, cyclic depsipeptides with 24 ring atoms (for example PF1022A) by means of fungal strains of the genus Rosellinia and further genera of the Xylariaceae family or by means of the enzymatic preparations isolated from these fungal strains. PF1022A, of the general formula (I), is outstandingly suitable for controlling endoparasites, in particular in the field of medicine and veterinary medicine.

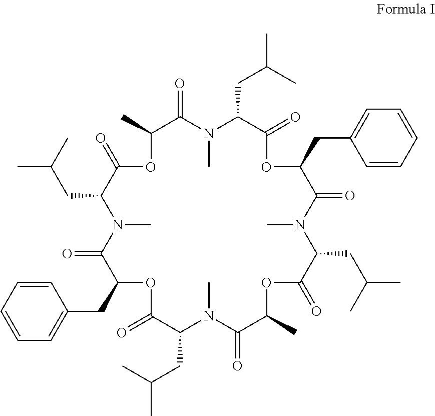

The present invention relates to a novel process for the preparation of lactic- and phenyl-lactic-acid-containing cyclic depsipeptides with 24 ring atoms. Such cyclic depsipeptides with 24 ring atoms (octadepsipeptides) and their use as endoparasiticides are already the subject matter of an earlier patent application (US005571793A). There is a series of chemical and microbial processes for the preparation of cyclic, D-2-hydroxyisovaleric-acid-containing depsipeptides with 24 ring atoms (for example by synthesis, cf.: Ohyama M. et al., Biosci. Biotechnol. Biochem. 58 (1994) pp. 1193-1194; Scherkenbeck. J. Et al. Tetrahedron 51 (1995) pp. 8459-8470; Kobayashi M. et al. Annu. Rep. Sankyo Res. Lab. 46 (1994) pp. 67-75; Lee B. et al. Bioorg. Med. Chem. Lett. 12 (2002) pp. 353-356; Dutton FE et al. J. Antibiot. 47 (1994) pp. 1322-1327).

The fermentation of the D-lactic- and D-phenyllactic-acid-containing cyclooctadepsipeptide PF1022A with the aid of a fungal strain, which has been referred to as “strain PF1022A”, is described in a Japanese patent (Sasaki T et al. J. Antibiot. 45 (1992) pp. 692-697; Yanai K et al. Nature Biotechnology 22 (2004) pp. 848-855). This strain PF1022A has been isolated from plant leaves of Camellia japonica, which had been collected in Japan in Ibaraki Prefektur. The strain was deposited at the “National Institute of Bioscience and Human Technology, Agency of Industrial Science and Technology” (Japan) as the Accession Number FERM BP-2671 and at the “Institute for Fermentation, Osaka” as No. IF033096. The PF1022A-producing fungal strain described by Meiji Saika Kaisha Ltd. belongs to the Xylariaceae family, its closest relatives are Rosellinia necatrix IFO 32537 (Miyadoh S. et al. Nippon Kingakukai Kaiho 41 (2000), pp. 183-188) and Xylaria polymorpha IFO 9780 (Sasaki Tet al. J. Antibiot. 45 (1992), pp. 692-697). These two strains do not produce any PF1022A compound.

The present invention relates to the finding of novel fungal strains which are isolated from fruiting bodies of Rosellinia spp. growing on dead wood of deciduous and coniferous trees, and further Xylariaceae, for the production of PF1022A.

Description of the isolation of fungal strains from fruiting bodies of Rosellinia spp. growing on dead wood of deciduous and coniferous trees, and further Xylariaceae, as potential active-substance formers of PF1022A:

The ubiquitous genus Rosellinia is assigned to the family Xylariaceae, which belongs to the extensive order Xylariales (phylum ascomycota). Representatives of this order have characteristic spherical fruiting bodies (perithecia) with defined ostioles, in which the asci with spores develop.

Besides the genus Rosellinia, the further genera Daldinia, Hypoxylon, Poronia, Ustulina and Xylaria are assigned to this family. Each of these genera has a series of different species within which producers of cyclic depsipeptides may potentially occur.

The family Xylariaceae has very hard brittle perithecia in which there are located the asci with in each case 8 dark brown to black spores. The genus Rosellinia comprises a series of species which live saprophytically or endophytically, but also manifest themselves as pathogens. Pathogenic species parasitize live timber and root systems of deciduous and coniferous trees. Saprophytic representatives of this genus live mainly on dead wood which is already undergoing decomposition processes. In nature, the individual, species have relatively high demands as regards the temperature, humidity and light regime. Their occurrence is additionally linked to the seasonal rhythm (in particular late winter, early spring), and to a high degree of undisturbedness of the preferred biotopes. Rosellinia species can be found in forests affected by little management, if any.

The fungal strains from the genus Rosellinia which are of interest here have all been isolated from samples of dead deciduous timber (for example sycamore, acacia). However, a precise attribution of the tree species to the respective dead timber sample is not always possible due to the varying degrees of decomposition.

Pure cultures were: obtained from mature, intact and, if possible, isolated perithecia removed from dead timber. They were surface disinfected with 0.05% strength AgNO₃ solution and rinsed repeatedly with sterile water. The perithecia were carefully squashed on a microscopic slide, and the asci and spores released were transferred into tubes containing sterile water.

By viewing under the microscope, it was possible to estimate the density and maturity stage at which the spores occurred. In the case of high spore densities, the spore suspension was subjected to decimal dilution, and 100 μl of, each dilution step Were plated onto malt extract agar (MEA). The plates were stored at 18 to 20° C., scored daily under the microscope, and germinating spores were transferred to fresh potato dextrose agar (PDA). The mycelium formed on this medium grows in the form of a shallow delicate whitish structure without the development of an aerial mycelium.

After the cultures had grown sufficiently, the mycelium was extracted with methanol, following, standard methods. The identification of the mycelium extract constituents and the quantification of the PF1022A contents were performed by means of LC-PDA-ESI-Q-TOF-MS and -MS/MS. Different Rosellinia lines were found, which proved: to be positive with regard to the formation of PF1022A.

Six PF1022A producers are assigned to the following species, according to morphological features:

Ja9=Rosellinia aquila of deciduous timer (more detailed determination not possible)

Ja15=Rosellinia aquila, from sycamore

Ja16=Rosellinia sp. from deciduous timber (more detailed determination not possible)

Ja19=Rosellinia aquila or R. corticum, either from acacia or from sycamore

Ja21=R. corticum of deciduous timer (more detailed determination not possible)

Ja26=Rosellinia sp. of deciduous timer (more detailed determination not possible)

Cultivation experiments were carried out. In comparison with the Mycelia sterilia T38-18 “wild-type” and strains Rosellinia abscondita CBS 448.89 and CBS 450.89, the newly-isolated fungal strains were found to show considerably more rapid growth on the media MEA, mPDA, CMA and seed agar.

The cultures Rosellinia aquila Ja-9, R. aquila Ja-15 and Rbsellinia sp. Ja-16 grew within 10 to 12 days at 21 to 22° C. up to the edge of the culture dish (9 cm), whereas the comparative cultures Mycelia sterilia T38-18 “wild-type”, Rosellinia abscondita CBS 448.89 and R. abscondita CBS 450.89 featured noticeably shorter diameters. This means that the novel Rosellinia strains feature much, more rapid growth. Strains Rosellinia aquila or R. corticum Ja-19, R. corticum Ja-21 and Rosellinia sp. Ja-26 grew equally rapidly.

A delicate mycelium developed on MEA and mPDA, while a dense and in some cases matted mycelium was formed on the high-nutrient media CMA and seed agar. On M1 medium, isolates Ja-19 and Ja-26 developed a mycelium which was partly plumate in appearance.

Composition of the media used:

CMA: maize meal 50 g/l, agar 15 g/l

M1: malt extract 10 g/l, yeast extract 4 g/l, glucose 4 g/l, agar 15 g/l

Seed agar: Pharmamedia 20 g/l, soyapeptone 2 g/l, maltose 40 g/l and MgSO₄×7 H₂O 2 g/l, NaCl 2 g/l, CaCO₃ 3 g/l, agar 15 g/l

MEA: malt extract 17 g/l, agar 15 g/l

mPDA: glucose 5 g/l, potato starch 20 g/l, yeast extract 1.5 g/l, agar 15 g/l

Since the present cultures are isolates which have been transferred directly from the natural ecosystem to artificial conditions, cultivars may occur which develop different mycelial shapes and a variation with regard to the intensity of active substance formation.

Description of the identification of the constituents, in particular PF1022A, in the mycelial extracts of the newly-isolated strains in comparison with Mycelia sterilia T38-18 (WT) by means of LC-PDA-ESI-Q-TOF-MS and -MS/MS:

To identify the constituents, in particular the PF1022 compounds, the methanolic mycelial extracts of Mycelia sterilia T38-18 (WT: wild type) and of the novel isolates Ja-9, Ja-15, Ja-16, Ja-19, Ja-21 and Ja-26 were studied by means of high-performance liquid chromatography (HPLC) using two detectors, photodiode array (PDA) and mass spectrometer (MS).

This study permits a comparison of the constituents' in the methanolic mycelial extracts (on seed agar medium) of Mycelia sterilia T38-18 (WT) with those of the novel isolate Ja-21 to be carried out with the aid of the HPLC chromatogram upon detection with PDA and MS detectors. It reveals a pronounced difference between the abovementioned strains in the production of the constituents, as follows:

Mycelia sterilia (WT) produces a series of PF1022 compounds: PF1022-A (C₅₂H₇₆N₄O₁₂), PF1022-B (C₆₄H₈₄N₄O₁₂), PF1022-C (C₅₈H₈₀N₄O₁₂), PF1022-D (C₄₆H₇₂N₄O₂), PF1022-E (C₅₂H₇₆N₄O₁₃) and PF1022-F (C₄₀H₆₈N₄O₁₂). Of these, PF1022-A (retention time Rₜ: 25.1 to 25.6 min in the chromatograms upon detection with PDA and MS detectors) and PF1022-D (Rₜ: 19.5 to 20 min) are the main constituents, while the novel isolates, for example strain Ja-21, besides

PF1022-A (characterization by means of LC-PDA-MS with the aid of UV/Vis and MS data and Rₜ, identical molecular weight with the same retention time in the HPLC chromatograms in comparison with PF1022A from Mycelia sterilia) and other compounds (Rₜ: 12 to 24 min), which do not belong to the PF1022 compounds, as the main constituents, also produces PF1022-B and -C as secondary compounds, but virtually no PF1022-D. Compounds PF1022-E and -F were not found here, either.

Similar results can also be found when comparing the TIC-MS chromatogram of Mycelia sterilia (WT) with those of the novel isolates Ja-15 and Ja-19. Further studies into the identification of the PF1022A compound were performed by means of LC-ESI-Q-TOF-MS/MS.

A comparison of the selected peaks of the PF1022A compound in the LC-MS/MS chromatograms of the mycelial extracts of Mycelia sterilia (WT) and of the novel isolates Ja-15 and Ja-19 was also carried out. These peaks have the same retention time. The MS/MS fragments of mass 949.6 of the proton adduct of the PF1022A compound from the mycelium extracts of Mycelia sterilia (WT) and of the novel isolates Ja-15 and Ja-19, which fragments were detected in the MS/MS spectra, are identical with one another.

The above results of the LC-PDA-ESI-Q-TOF-MS and -MS/MS studies confirm that the novel isolates such as Ja-15, Ja-19 and Ja-21 and also Ja-9, Ja-16 and Ja-26 are not Mycelia sterilia (WT), but also produce the PF1022A compound.

Since the novel Rosellinia and Xylaria strains do not produce the secondary products PF1022-D, -E and -F, the method of extracting and isolating PF1022A is greatly simplified in comparison with Mycelia sterilia (WT).

1. A process for the preparation of PF1022A, of the formula 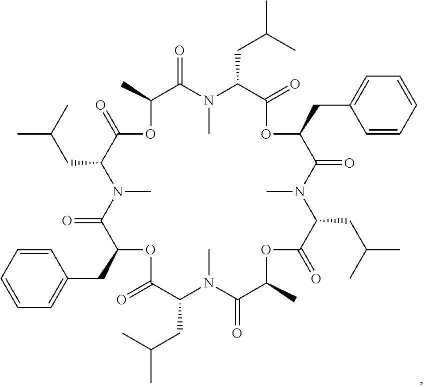
a cyclic octadepsipeptide with 24 ring atoms, with the aid of fungal strains of the species Rosellinia and further genera of the family Xylariaceae or by means of the enzymatic preparations isolated from these fungal strains.

2. The process according to claim 1, with the aid of the fungal strains Rosellinia spp. and Xylariacea.

3. The process according to claim 2, wherein the Rosellinia spp. is selected from the group consisting of Rosellinia aquila or Rosellinia corticum.
